(19) United States
(12) Patent Application Publication
(10) Pub. No.: US 2010/0280418 A1
(43) Pub. Date: Nov. 4, 2010

(54) METHOD AND SYSTEM FOR EVALUATING A MOVEMENT OF A PATIENT

(76) Inventor: Hans-Peter KLOSE, Stuttgart (DE)

Correspondence Address: KENYON & KENYON LLP, ONE BROADWAY, NEW YORK, NY, 10004, US

(21) Appl. No.: 12/730,035

(22) Filed: Mar. 23, 2010

(30) Foreign Application Priority Data
Apr. 30, 2009 (DE) 102009002747.5

(51) Int. Cl. A61B 5/103

(52) U.S. Cl. 600/595

(57) ABSTRACT

A system for evaluating the movement of a patient having a first display unit for displaying a reference movement, a first detection unit, to be situated in particular in the location of the patient, for detecting the movement of the patient, an evaluation unit for ascertaining a measure characterizing a deviation between the detected movement and the reference movement, which is finally displayed on the first or a second display unit. It is possible to achieve an evaluation of the movement of the patient that is independent of the presence of a therapist.

### BACKGROUND INFORMATION

In many diseases, extensive rehabilitation measures are required today after the occurrence of an acute event. Following a stroke for example, a great number of patients must relearn the simplest motor skills. Typical concomitant phenomena of a stroke are hemiplegias, which result from damage to areas of the brain in which these abilities are located. Through protracted training, however, it is possible to relearn and often completely restore the failed functions.

A very frequently used method is kinesitherapy under the guidance of trained physiotherapists/occupational therapists in the context of in-patient rehabilitation. In addition, there is the option of telerehabilitation, which may be used following the in-patient rehabilitation—in less severe cases possibly even directly after discharge from acute treatment. For this purpose, normally a real-time video transmission is used between the patient (at his own home) and the therapist (in his practice). The therapist may demonstrate exercises for example, which are transmitted by video to the patient. The patient reproduces these. This is likewise transmitted to the therapist, who observes and evaluates the execution. German Patent Application No. DE 10 2004 001 426 describes a respective method, which makes it possible for a therapist to evaluate the movement of a patient in a spatially and temporally independent manner. These methods, however, require much personnel and time and are thus expensive.

It is thus desirable to simplify the evaluation of the movement of a patient and in particular to make it temporally and spatially independent of the presence of a therapist.

Japanese Patent No. JP 2006-116325 describes a state analyzer for a medical treatment, which ascertains the activity of a person such as e.g. sitting, lying, walking, standing, etc. during medical treatment by comparing averaged sensor values with respective threshold values. The activity may be recorded, analyzed and evaluated. The sensor is used to prepare a movement profile of the patient.

PCT International Patent Publication No. WO 01/007112 shows a system for rehabilitation comprising a muscle contraction sensor for controlling a computer game.

### SUMMARY OF THE INVENTION

The present invention provides in particular the detection of the movements of a (rehabilitation) patient, the comparison with one or more reference movements predefined by the therapist, and their objective evaluation by deviation analysis. This includes in particular the evaluation of a deviation from a final position, the evaluation of the speed of the execution, the evaluation of the uniformity of the movement, etc. This makes it possible to calculate a corresponding objectifiable measure. This may be detected for example in the context of telemonitoring and be documented for the therapist. This measure may be represented in particular as a number, surface area or the like.

This technology makes it possible to relieve the therapists since patients are able to exercise independently without constant monitoring on the part of the therapist. The therapist is thus able to attend to more patients, which, in light of the expected rate of increase in strokes for example (projection: +50-70% by 2050) and the decreasing number of available specialists, reduces costs and is advantageous for maintaining medical care. Problems with providing care, particularly in rural areas, are more readily solvable. The therapist receives quantitative data about the progress of the rehabilitation, which makes it possible to optimize the individual rehabilitation program. The patient is able to exercise independently with qualitative and/or quantitative feedback. The provided system may be used to train individual extremities (e.g. left arm, left leg), muscle groups (e.g. upper body, arms, legs), fine motor skills (e.g. hands, fingers), etc.

The technical implementation of the present invention in particular makes use of the fact that movements may be detected for example by video sensors (e.g. movement analysis for athletes) and/or inertial sensors (e.g. tool robots) and may be evaluated by image analysis or analysis of the acceleration components (lateral and angular accelerations) using software. The evaluations allow for a realistic transference to virtual, animated persons. Such systems and methods are known for example by the catchword “motion tracking”. For this purpose it is useful to be able to map the basic movements onto a kind of rod puppet and its joint points.

This movement detection may be performed both in patients (e.g. the actual movement of the left arm) and in physiotherapists (e.g. specifying the target movement of the left arm). If both movements are mapped onto each other by suitable methods (e.g. scaling the length of the extremities), then a deviation between the target movement and the actual movement may be calculated and converted into a quantitative measure of the deviation. At the same time it is possible to detect how quickly or uniformly or jerkily the movement was performed. The deviation may be communicated to the patient by visualization on a screen, e.g. as a colored surface.

A refinement provides for a memory unit for storing the detected movement of the patient and/or the ascertained measure. This allows in particular for a transmission to a therapist station, which allows the therapist to check the movement. This may occur at regular intervals so that it is possible to detect a progress in the therapy. The stored data may also be used for analysis purposes at a later time. The evaluation of the movement on the part of a therapist or the like may occur in particular separated in time from the performance on the part of the patient.

Particularly advantageously, a third display unit, to be situated in particular in the location of the therapist, is provided for displaying the detected movement and/or the ascertained measure. The evaluation of the movement on the part of a therapist or the like may thus occur in particular in spatial separation from the performance of the patient. The display for the therapist may in particular also include ascertained numerical values for reference characteristics.

Expediently, the first and/or the second detection unit have a video sensor system unit. For characterizing the recorded movement, the image signals are evaluated accordingly using suitable software. There may be a provision to attach readily detectable markings, e.g. reflecting dots, at defined positions on the body. Advantageously, support may also be provided by special clothing. In this manner, a movement may be characterized simply and reliably.

Equally advantageously, the first and/or the second detection unit have an inertial sensor system unit. When using inertial sensors, these should be fixed in place at defined locations on the patient/therapist. Various approaches are considered. A sensor sleeve may be provided for detecting an arm movement. The positioning may occur by suitably fixing the one sleeve opening (cuff) in place, e.g. by a strap over the thumb. The upper sleeve opening is fixed in place, e.g. by a holster-like strap on the opposite shoulder or on the neck (e.g. a strap having a Velcro fastener). The material is preferably a stretchable material, into which pockets are integrated for accommodating multiple sensors (e.g. on the wrist, elbow, forearm, upper arm, shoulder, etc.). The sensors should exchange the data preferably wirelessly with a receiver. The latter may also be accommodated in the sleeve or be developed as a separate device. The alternative to pockets may be e.g. Velcro points, on which the sensors are attached. The sensors may also be attached by Velcro bands at the defined locations around the arm. For detecting a hand movement, a development e.g. as a glove may be provided, sensors being provided, e.g. in the finger tips and the metacarpus. For detecting a leg movement, a development e.g. as a pant leg may be provided, which is fastened on the foot or the sole of the foot and the hip. The fastening may occur as explained above in connection with the sleeve. The sensors are expediently situated on the forefoot, the ankle joint, knee, thigh, lower leg, hip, etc.

A preferred specific embodiment includes a station to be situated at the patient's location and a station to be situated at the therapist's location, the stations being equipped for example as PCs with appropriate detection devices such as e.g. a camera and appropriate software. Alternatively, a portable system or hand-held device is conceivable for mobile patients having less severe limitations or patients who are already better trained, which guides the patient and provides feedback via appropriate symbols (e.g. two superposed stick figures or rod puppets).

### BRIEF DESCRIPTION OF THE DRAWINGS

FIG. 1 schematically shows a preferred specific embodiment of a system according to the present invention.

FIG. 2 schematically shows a flow chart of a preferred specific embodiment of a method according to the present invention.

### DETAILED DESCRIPTION

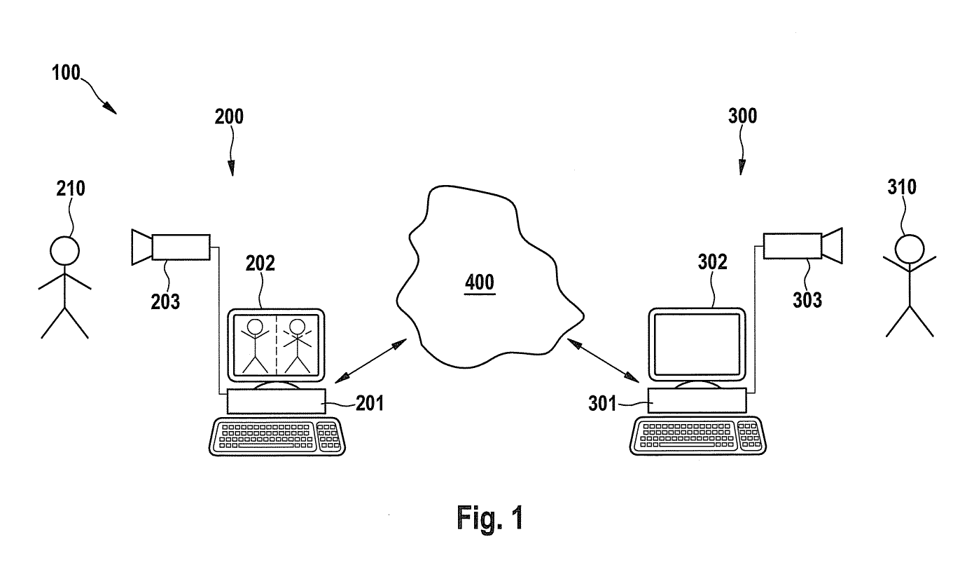

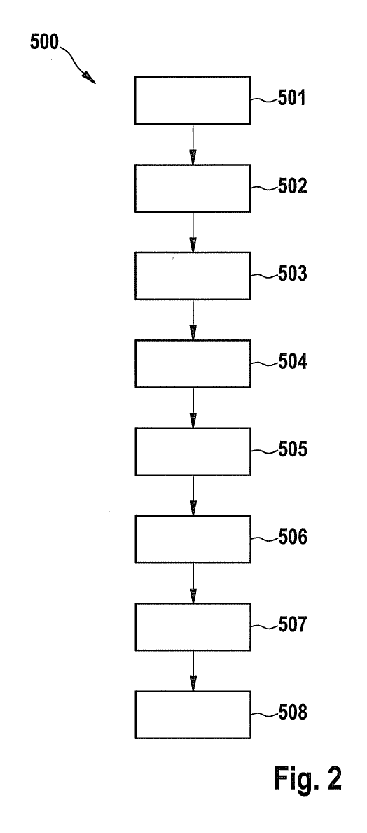

FIGS. 1 and 2 will be described in the following coherently and comprehensively, the specific embodiment of the method of the present invention shown in FIG. 2 being based on a system as shown in FIG. 1.

FIG. 1 schematically shows a preferred development of a system according to the present invention and indicated as a whole by 100. System 100 has a station 200 to be situated at the location of a patient 210 and a station 300 to be situated at the location of a therapist 310. In the chosen example, both stations 200 and 300 are developed as computers 201 and 301, respectively having one screen 202 and 302 and respectively one video camera 203 and 303. These stations 200 and 300 are connected via a telecommunications network 400, for example the Internet. Depending on the design of the telecommunications network and the possible access to it by third parties, encryption and/or authentication methods according to the related art are provided in order to secure the communication between the medical device and the remote unit. As a result of the spatially and temporally separated evaluation option offered by the present invention, however, it is also possible for the two stations not to be connected and for the data instead to be exchanged by diskettes, CDs, flash memories and the like.

In FIG. 2, a preferred development of a method according to the present invention is represented as a flow chart and indicated as a whole by 500. The method begins in a method step 501, in which an operator, for example physiotherapist 310, performs a reference movement at station 300, which is detected by a second detection unit developed here as a video camera 303.

In a method step 502, the recorded reference movement is analyzed, reference characteristics such as e.g. joint positions, joint angles, speeds, etc. being determined in the process. In particular, suitable software in computer 301 may be used for this purpose.

In a subsequent method step 503, the record of the reference movement and/or the reference characteristics are transmitted to station 200 situated in the location of patient 210.

In a method step 504, the recorded reference movement or a reference movement generated on the basis of the reference characteristics (“rod puppet”) is presented to the patient on the first display unit developed here as a screen 202. The presentation of the reference movement may include both a video recording of the therapist himself as well as a virtual person, e.g. a rod puppet.

Independently of and outside of the present invention, the patient performs a movement to imitate the reference movement. This movement of the patient is detected in step 505 by the first detection unit developed here as video camera 203 and is supplied to computer 201.

Using suitable software, computer 201 ascertains in step 506 on the basis of a comparison, and taking into account the reference characteristics, a measure characterizing a deviation between the detected movement and the reference movement.

In a method step 507, the measure is displayed to patient 210 on screen 202 so that he is able to improve his sequence of movements if necessary. The display may include for example the movement of the patient superposed on the reference movement, the deviation being marked as colored surfaces for example. The reference movement and the movement of the patient may also be presented side-by-side for example.

In another method step 508, the detected movement of the patient is stored together with the characterizing measure in computer 201 and/or is transmitted to station 300 situated in the location of therapist 310 for later assessment.

What is claimed is:

1. A system for evaluating a movement of a patient, comprising:
at least one display unit for displaying a reference movement;
a first detection unit situated in a location of the patient for detecting a movement of the patient; and
an evaluation unit for ascertaining a measure characterizing a deviation between the detected movement and the reference movement,
wherein the at least one display unit displays the ascertained measure.

2. The system according to claim 1, further comprising a memory unit for storing at least one of the detected movement of the patient and the ascertained measure.

3. The system according to claim 1, further comprising a second detection unit situated in a location of a therapist for detecting the reference movement.

4. The system according to claim 1, further comprising a further display unit situated in a location of a therapist for displaying at least one of the detected movement and the ascertained measure.

5. The system according to claim 3, wherein at least one of the first and second detection units includes a video sensor system unit.

6. The system according to claim 3, wherein at least one of the first and second detection units includes an inertial sensor system unit.

7. The system according to claim 1, further comprising:
a first station situated in the location of the patient; and
a second station situated in a location of a therapist.

8. A method for evaluating a movement of a patient, comprising:
displaying a reference movement;
detecting the movement of the patient;
ascertaining a measure characterizing a deviation between the detected movement and the reference movement; and
displaying the ascertained measure.

9. The method according to claim 8, further comprising detecting the reference movement.

10. The method according to claim 8, further comprising storing at least one of the movement of the patient and the ascertained measure.
